(19) United States
(12) Patent Application Publication
(10) Pub. No.: US 2015/0223973 A1
(43) Pub. Date: Aug. 13, 2015

(54) WEARER CONTROLLED BODY TEMPERATURE REGULATION DEVICE

(71) Applicant: Stuart Frederick, Naples, FL (US)

(72) Inventor: Stuart Frederick, Naples, FL (US)

(21) Appl. No.: 14/178,451

(22) Filed: Feb. 12, 2014

(51) Int. Cl. A61F 7/00

(52) U.S. Cl. CPC A61F 7/0085; A61F 2007/0009; A61F 2007/006; A61F 2007/0064

(57) ABSTRACT

A body temperature regulation device controlled by a wearer comprising a housing, where the housing is positioned by the wearer's neck; a compressor within the housing, where the compressor pumps hot and cold air as selected by the wearer; a plurality of vents within the housing, where the plurality of vents enable the hot and cold air to escape the housing; a neck piece attached to the housing, where the neck piece wraps around the neck; and an internal tubing within the neck piece, where the internal tuning receives the hot and cold air from the compressor to circulate the hot and cold air around the wearer's neck.

### BACKGROUND OF THE INVENTION

1. Field of the Invention

The present invention relates to a device worn around a user's neck to circulate warm or cold air as needed by the wearer.

2. Description of Related Art

A person's body temperature can fluctuate drastically when exposed to extremely hot or cold temperatures. The body temperature elevates when the body produces or absorbs more heat than it can dissipate. This is especially prevalent in athletes, who exert so much energy, that it is often difficult to bring their temperature down without assistance. Likewise, individuals who live in very warm climates often have trouble cooling themselves when exposed to the hot sun or high humidity. Sweating is the body's natural mechanism to cool the body, and when combined with external forces like a cold beverage, fan or air conditioning the person is able to maintain a comfortable body temperature. But if the temperature exceeds a comfortable level physical ailments like hyperthermia or heat stroke can occur which can sometimes lead to death.

On the opposing end of the spectrum hypothermia is a condition where a person's body temperature drops below a point necessary for normal body functions. Hypothermia can cause limb amputation due to loss of blood flow to the extremities when the body is trying to ensure that the vital organs stay functioning or even death in the most extreme cases. Before this condition sets in the user will feel cold, start to shiver and experience mental confusion. To prevent the onset of hypothermia the person must ensure that they are wearing appropriate clothing like wool fabrics or extra layers of undergarments, covering their head or staying in a shelter to shield from extreme winds and cold. Even with these precautions hypothermia may begin when the temperatures and wind are especially harsh.

In light of the aforementioned conditions, it would be beneficial in the art to provide a device that assists a user to maintain their normal body temperature when faced with extreme hot or cold temperatures. It would also be desirable in the art to provide a single device that can be used in both extremes to cool or warm as needed by the user.

### SUMMARY OF THE INVENTION

In view of the foregoing disadvantages inherent in the prior art, the general purpose of the present invention is to provide a device to regulate a user's body temperature, configured to include all of the advantages of the prior art, and to overcome the drawbacks inherent therein.

Accordingly, an object of the present invention is to provide a body temperature regulation device to enable a wearer to control their body temperature when exposed to harsh weather.

Another object of the present invention is to provide a body temperature regulation device that blows hot or cold air through vents within the device.

Yet another object of the present invention is to provide a body temperature regulation device that further circulates hot or cold air through a tube circled around the user's neck.

To achieve the above objects, in an aspect of the present invention, a body temperature regulation device is described controlled by a wearer comprising a housing, where the housing is positioned by the wearer's neck; a compressor within the housing, where the compressor pumps hot and cold air as selected by the wearer; a plurality of vents within the housing, where the plurality of vents enable the hot and cold air to escape the housing; a neck piece attached to the housing, where the neck piece wraps around the neck; and an internal tubing within the neck piece, where the internal tuning receives the hot and cold air from the compressor to circulate the hot and cold air around the wearer's neck.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing in which:

FIG. 1 depicts a perspective view of a body temperature regulation device in accordance with an exemplary embodiment of the present invention.

### DETAILED DESCRIPTION OF THE DRAWING

The present invention relates to a device worn around a user's neck to circulate warm or cold air as needed by the wearer. The present invention provides a body temperature regulation device to enable a user to control their body temperature thereby providing comfort to the user when facing extremely hot or cold weather. The body temperature regulation device includes a temperature regulator worn around the user's neck. The temperature regulator is insulated and emits warm or cool air as preferred by the wearer. The temperature regulator includes small air vents to circulate the treated air, thereby maintaining the user's core body temperature even while in extreme weather.

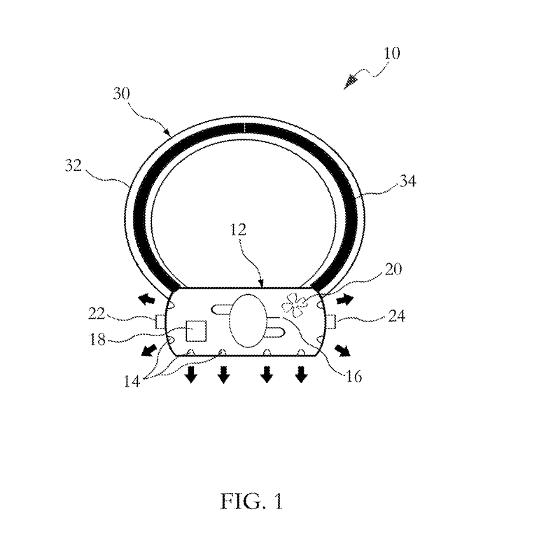

Turning now descriptively to the drawing, referring to FIG. 1, a body temperature regulation device 10 is shown in accordance with an exemplary embodiment of the present invention. The body temperature regulation device 10 includes a housing 12 that pumps hot or cold air as preferred by a user. The housing 12 includes a plurality of vents 14 through which hot or cold air is passed. The hot/cold air is created by a compressor 16 within the housing 12. The housing 12 further includes a battery compartment 18 to house the batteries required to power the body temperature regulation device 10. The batteries may be lithium batteries. An activation button 22 fastened on the surface of the housing 12 may be coupled to the compressor 16 and the batteries to initiate the flow of hot/cold air. Likewise, a temperature control dial 24 may be coupled to the compressor 16 to allow the user to select between creating hot or cold air by the compressor 16. Once the hot/cold air is created by the compressor 16 a small fan 20 circulates the air through the housing 12 and out through the vents 14, as illustrated with the arrows.

Fastened to the housing 12 is a neck piece 30. The neck piece 30 may be a necklace, wrap, scarf or the like. The neck piece 30 is covered with an insulating material 32 which is wrapped around and completely envelops an internal tubing 34. The insulating material 32 ensures that the internal tubing 34 maintains a preferred temperature. The insulating material 32 may be neoprene, foam, down, synthetic fibers, and the like. The internal tubing 34 receives the pumped air from the housing 12 to circulate the hot/cold air through the neck piece 30 thus maintaining a selected temperature around the user's neck. Along with pumping the hot/cold air through the housing 12 and the vents 14, the small fan 20 pumps the air through the internal tubing 34 as well.

The body temperature regulation device 10 circulates and releases hot/cold air as selected by the user. The device 10 is especially useful when the user is exposed to extreme temperatures thereby preventing adverse body ailments due to harsh weather or over exertion. By positioning the body temperature regulation device 10 around the user's neck, their body temperature may be quickly controlled because of the close proximity the device 10 has to the user's major veins and arteries in the neck. This allows the blood flowing through these passages to quickly warm or cool thereby circulate that altered blood through the rest of the user's body.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A body temperature regulation device controlled by a wearer comprising:
a. a housing, where the housing is positioned by the wearer's neck;
b. a compressor within the housing, where the compressor pumps hot and cold air as selected by the wearer;
c. a plurality of vents within the housing, where the plurality of vents enable the hot and cold air to escape the housing;
d. a neck piece attached to the housing, where the neck piece wraps around the neck; and
e. an internal tubing within the neck piece, where the internal tuning receives the hot and cold air from the compressor to circulate the hot and cold air around the wearer's neck.

2. The body temperature regulation device according to claim 1, where the neck piece is one of at least a necklace, wrap or scarf.

3. The body temperature regulation device according to claim 1, where the housing includes a fan, where the fan circulates the hot and cold air through the plurality of vents and the internal tubing.

4. The body temperature regulation device according to claim 1, where the housing includes a battery compartment.

5. The body temperature regulation device according to claim 1, where the battery compartment receives a set of lithium batteries.

6. The body temperature regulation device according to claim 1, where the housing includes an activation button coupled to the battery compartment and compressor.

7. The body temperature regulation device according to claim 1, where the housing includes a temperature control dial, where the temperature control dial enables the user to select between hot and cold air circulating through the plurality of vents and the internal tubing.

8. The body temperature regulation device according to claim 1, where the neck piece includes an insulating cover, where the insulating cover envelopes the internal tubing.
